(19) United States
(12) Patent Application Publication
(10) Pub. No.: US 2022/0387408 A1
(43) Pub. Date: Dec. 8, 2022

(54) USE OF MAST CELL STABILIZER FOR THE TREATMENT OF HEART FAILURE WITH PRESERVED EJECTION FRACTION

(71) Applicants: INSERM (INSTITUT NATIONAL DE LA SANTÉ ET DE LA RECHERCHE MÉDICALE), Paris (FR); UNIVERSITÉ DE BORDEAUX, Bordeaux (FR); CHU DE BORDEAUX, Talence (FR)

(72) Inventors: Marie-Ange RENAULT, Pessac (FR); Thierry COUFFINHAL, Pessac (FR); Candice CHAPOULY, Pessac (FR); Sarah GUIMBAL, Pessac (FR)

(21) Appl. No.: 17/776,075

(22) PCT Filed: Nov. 10, 2020
(86) PCT No.: PCT/EP2020/081607

§ 371 Date: May 11, 2022

(30) Foreign Application Priority Data
Nov. 12, 2019 (EP) 19208649.4

(51) Int. Cl. A61K 31/4422; A61K 31/352; A61K 31/4545; A61K 31/495; A61K 31/506; A61K 31/445; A61P 9/04

(52) U.S. Cl. CPC A61K 31/4422; A61K 31/352; A61K 31/4545; A61K 31/495; A61K 31/506; A61K 31/445; A61P 9/04

(57) ABSTRACT

Heart failure with preserved ejection fraction (HFpEF) which results from diastolic dysfunction is a growing epidemiologic problem. However, the pathophysiology of this disease is poorly understood. Our goal is to investigate whether microvessel disease may promote HFpEF. To do so we have used Leptin receptor deficient (Lepr^{db/db}) female mice as a model of HFpEF and performed a transcriptomic analysis via RNA sequencing of the cardiac vascular fraction of both these mice and their control Lepr^{db}/+littermates. In Lepr^{db/db} female mice, end diastolic pressure (EDP) signing diastolic dysfunction is significantly increased from 3 month of age. It is correlated with a cardiac and cardiomayocyte hypertrophy, vascular leakage, endothelial cell activation and leucocyte infiltration. As expected, the RNA sequencing analysis confirmed endothelial dysfunction. Besides, it also revealed a strong increase in several mast cell markers. We confirmed, via histology, an accumulation of mast cells in the heart of Lepr^{db/db} mice. Importantly, it was associated with increased levels of circulating IgE. Lepr^{db/db} mice were then treated or not with Cromolyn sodium, an inhibitor of mast cell degranulation. After a month treatment, EDP was significantly reduced in Lepr^{db/db} mice demonstrating the critical role of mast cell in the development of diastolic dysfunction in diabetic obese mice.

### FIELD OF THE INVENTION

The present invention is in the field of medicine, in particular cardiology.

### BACKGROUND OF THE INVENTION

Heart failure with preserved ejection fraction (HFPEF), formerly called Diastolic Heart Failure (DHF), accounts for more than 50% of all heart failure cases and portrays a high risk of morbidity and mortality. Patients suffering from HFPEF is associated with a decline in diastolic performance of the left ventricle of the heart. When the cardiac muscle has become stiff and lost its ability to relax the left ventricle is not readily filled with blood following contraction and the cardiac output becomes either diminished or an elevated ventricular diastolic pressure despite essentially normal end diastolic volume (EDV) is observed for compensation. The HFPEF is often characterized histologically by a hypertrophy of cardiomyocytes, increased interstitial collagen deposition and calcium deposition within the myocardium which are assumed to lead collectively to decreased distensibility and compliance. The chemo-mechanical characteristics of the heart muscle proteins as well as myocytes and the biophysics of the failing heart have not yet achieved clinical relevance. There is no specific treatment of HFPEF available. When the chronic condition is tolerable by the patient, the therapy may be directed at aggravating factors such as high blood pressure and diabetes. Diuretics are often given. The administration of calcium channel and/or angiotensin II receptor blocker drugs may be of benefit in reducing ventricular stiffness in some cases but there is no favorable effect in mortality rates. A major complication is pulmonary edema the treatment of which by diuretics is often challenging since the stiffened heart and vessels of the patients are very susceptible to hypotensive events after salt and water excretion. Thus, there is an unmet medical need for the treatment of HFPEF.

### SUMMARY OF THE INVENTION

As defined by the claims, the present invention relates to use of mast cell stabilizer for the treatment of heart failure with preserved ejection fraction.

### DETAILED DESCRIPTION OF THE INVENTION

The present invention relates to a method of treating heart failure with preserved ejection fraction (HFPEF) in a subject in need thereof comprising administering to the subject a therapeutically effective amount of a mast cell stabilizer.

As used herein, the term “heart failure with preserved ejection fraction” has its general meaning in the art and refers to a complex syndrome characterized by heart failure (HF) signs and symptoms and a normal or near-normal left ventricular ejection fraction (LVEF). More specific diagnostic criteria include signs/symptoms of HF, objective evidence of diastolic dysfunction, disturbed left ventricular (LV) filling, structural heart disease, and elevated brain natriuretic peptides. Additional cardiac abnormalities can include subtle alterations of systolic function, impaired atrial function, chronotropic incompetence, or haemodynamic alterations, such as elevated pre-load volumes.

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a patient having a medical disorder or who ultimately may acquire the disorder, in order to prevent, cure, delay the onset of, reduce the severity of, or ameliorate one or more symptoms of a disorder or recurring disorder, or in order to prolong the survival of a patient beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

As used herein, the term “mast cell” refers to a bone marrow derived cell that mediates hypersensitivity reactions. Mast cells are characterized by the presence of cytoplasmic granules (histamine, chondroitin sulfate, proteases) that mediate hypersensitivity reactions, high levels of the receptor for IgE (FceRI), and require stem cell factor and IL3 (cytokines) for development. Mature mast cells are not found in the circulation, but reside in a variety of tissues throughout the body.

As used herein, a “mast cell stabilizer” refers to an agent that inhibits degranulation and/or the release of pro-inflammatory and vasoactive mediators from mast cells.

In some embodiment, mast cell stabilizers include, but are not limited to, cromolyn, dihydropyridines such as nicardipine and nifedipine, lodoxamide, nedocromil, barnidipine, YC-114, elgodipine, niguldipine, ketotifen, methylxanthines, quercetin, and pharmaceutically salts thereof. In some embodiments, the mast cell stabilizer is a pharmaceutically acceptable salt of cromolyn, such as cromolyn sodium, cromolyn lysinate, ammonium cromonglycate, and magnesium cromoglycate. In some embodiments, mast cell stabilizers include but are not limited to compounds disclosed in U.S. Pat. Nos. 6,207,684; 4,634,699; 6,207,684; 4,871,865; 4,923,892; 6,225,327; 7,060,827; 8,470,805; 5,618,842; 5,552,436; 5,576,346; 8,252,807; 8,088,935; 8,617,517; 4,268,519; 4,189,571; 3,790,580; 3,720,690; 3,777,033; 4,067,992; 4,152,448; 3,419,578; 4,847,286; 3,683,320; and 4,362,742; U.S. Patent Application Publication Nos. 2011/112183 and 2014/140927; European Patent Nos. 2391618; 0163683; 0413583; and 0304802; International Patent Application Nos. WO2010/042504; WO85/02541; WO2014/115098; WO2005/063732; WO2009/131695; and WO2010/088455; all of which are incorporated by reference. Mast cell stabilizers, including cromolyn and pharmaceutically acceptable salts, prodrugs, and adducts thereof, may be prepared by methods known in the art.

In some embodiment, mast cell stabilizers are antihistamines. Example of antihistamine include, but are not limited to azatadine, cetirizine, mizolastine, and/or newer-generation drugs such as desloratadine, fexofenadine, and levocetirizine.

As used herein, the term “antihistamine” refer to drugs which treat allergic rhinitis and other allergies.

By a “therapeutically effective amount” of the mast cell stabilizer of the invention as above described is meant a sufficient amount of the compound. It will be understood, however, that the total daily usage of the compounds and compositions of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidential with the specific polypeptide employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Preferably, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

The mast cell stabilizer of the invention may be combined with pharmaceutically acceptable excipients, and optionally sustained-release matrices, such as biodegradable polymers, to form therapeutic compositions. “Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. In the pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms. Galenic adaptations may be done for specific delivery in the small intestine or colon. Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol ; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions comprising mast cell stabilizers of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. The mast cell stabilizer of the invention can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifusoluble agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. Sterile injectable solutions are prepared by incorporating the active polypeptides in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject. The mast cell stabilizer of the invention may be formulated within a therapeutic mixture to comprise about 0.0001 to 1.0 milligrams, or about 0.001 to 0.1 milligrams, or about 0.1 to 1.0 or even about 10 milligrams per dose or so. Multiple doses can also be administered. In addition to the mast cell stabilizers of the invention formulated for parenteral administration, such as intravenous or intramuscular injection, other pharmaceutically acceptable forms include, e.g. tablets or other solids for oral administration; liposomal formulations; time release capsules; and any other form currently used.

The invention will be further illustrated by the following figures and examples. However, these examples and figures should not be interpreted in any way as limiting the scope of the present invention.

### FIGURES

FIG. 1: Cromolyn Sodium therapy prevents the occurrence of diastolic dysfunction in Lepr^{db/db} mice. 2 month old Lepr^{db/db} female mice were either treated with 50 mg/kg/day cromolyn sodium vs control vehicle or 4 mg/kg/day cetirizine vs control vehicle for 28 days. (A-B) At the end of the treatment end diastolic pressure was measured using a pressure catheter and compared to the one of 3 month old Lepr^{db}/+ control mice. At 3 month of age, mice were subjected to echocardiography, LV catheterization and sacrificed. End diastolic pressure was measured using a pressure catheter (n=9-15 mice per group).

FIG. 2: Cromolyn sodium decreases vascular permeability and leucocyte infiltration in Lepr^{db/db} female mice. 2 month old Lepr^{db/db} female mice were treated or not with 50 mg/kg/day cromolyn sodium for 28 days. Mice were sacrificed at 3 month of age. Mice were subjected to echocardiography, LV catheterization and sacrificed. (A) Capillary density was quantified as the number of CD31+ vessels/mm² (n=8 mice/group). (B) The mean cardiac capillary diameter was measured (n=8 mice/group). (C) Albumin extravasation was measured as the albumin+ surface area (n=8 mice/group). (D) Leucocyte infiltration was measured as the number of CD45+ cells/mm² (n=10 mice/group). (E) The mean cardiac capillary diameter was measured (n=10 and 7 mice/group respectively). (F) Albumin extravasation was measured as the albumin+surface area (n=10 and 7 mice/group respectively).*: p≤0.05; **: p≤0.01; ***: p≤0.001. NS: not significant. Mann Whitney test.

### EXAMPLE

Methods

Mice

Lepr^{db} mice (BKS.Cg-Dock7m/+ Lepr^{db/+}J) were obtained from Charles River laboratories and bred together to obtain Lepr^{db/db} and control Lepr^{db/+} mice.

Animal experiments were performed in accordance with the guidelines from Directive 2010/63/EU of the European Parliament on the protection of animals used for scientific purposes and approved by the local Animal Care and Use Committee of Bordeaux University. Only females were used. Mice were either sacrificed by cervical dislocation or exsanguination under deep anesthesia (ketamine 100 mg/kg and xylazine 20 mg/kg, IP).

Cromolyn Sodium/Cetirizine Therapy

To prevent mast cell degranulation, mice were treated with 50 mg/kg/day cromolyn sodium (Abcam) via intra-peritoneal injections for 28 days. Untreated mice received 0.9% NaCl daily intra-peritoneal injections. To investigate the role of Histamine release by mast cell, mice were treated with 4 mg/kg/day cetirizine (Arrow Generiques) orally (in the drinking water) for 28 days.

Echocardiography

Left-ventricular ejection fraction and LV dimension will be measured on a high-resolution echocardiographic system equipped with a 30-MHz mechanical transducer (VEVO 2100, VisualSonics Inc.) as previously described 13,14. Mice were anchored to a warming platform in a supine position, limbs were taped to the echocardiograph electrodes, and chests were shaved and cleaned with a chemical hair remover to minimize ultrasound attenuation. UNI′GEL ECG (Asept Inmed), from which all air bubbles had been expelled, was applied to the thorax to optimize the visibility of the cardiac chambers. Ejection fractions were evaluated by planimetry as recommended (Schiller et al. 1989). Two-dimensional, parasternal long-axis and short-axis views were acquired, and the endocardial area of each frame was calculated by tracing the endocardial limits in the long-axis view, then the minimal and maximal areas were used to determine the left-ventricular end-systolic (ESV) and end-diastolic (EDV) volumes, respectively. The system software uses a formula based on a cylindrical-hemiellipsoid model (volume=8·area²/3π/length) 15. The left-ventricular ejection fraction was derived from the following formula: (EDV−ESV)/EDV*100. The cardiac wall thickness (Left ventricular posterior wall (LVPW), Inter-ventricular septum (IVS) and left ventricular internal diameter (LVID) were calculated by tracing wall limits in both the long and short axis views.

LV Pressure/Systolic Blood Pressure Measurement

LV diastolic pressure measurement was assessed using pressure—volume conductance catheter technique. Briefly, mice will be anesthetized with Isoflurane. A Scisense pressure catheter (Transonic) will be inserted into the LV through the common carotid artery. Pressure will be recorded using (which was not certified by peer review) is the author/funder. All rights reserved. No reuse allowed without permission. LabChart software. End diastolic pressure, dP/dt minimum and maximum, Tau and heart rate were automatically calculated by a curve fit through end-systolic and end-diastolic points on the pressure plot.

Immuno-Histological Assessments

Prior to staining, heart were stopped in diastole using KCl, perfused and then fixed in 10% formalin for 4 hours, paraffin embedded and cut into 7 μm thick sections. Alternatively, heart were fresh frozen in OCT, then cut into 7 μm thick sections.

ECs were identified using rat anti-CD31 antibodies (Histonova, cat #DIA-310). Albumin was stained using sheep anti-albumin antibodies (Abcam, Cat #ab8940). Pan-leucocytes were identified using rat anti-mouse CD45 antibodies (BD Pharmingen Inc, Cat #550539).

For immunofluorescence analyzes, primary antibodies were resolved with Alexa Fluor®—conjugated secondary polyclonal antibodies (Invitrogen, Cat #A-21206, A-21208, A-11077, A-11057, A-31573, A-10037) and nuclei were counterstained with DAPI (1/5000). Negative controls using secondary antibodies only were done to check for antibody specificity.

Capillary density (CD31+ vessels) was quantified in 4 pictures taken under ×260 magnification in areas where cardiomyocytes were oriented transversally.

To assess the mean capillary diameter, the diameter of 10 capillary randomly chosen in each picture was measured via Image J. Inflammatory cell density (CD45+) was quantified in 8 pictures randomly taken under ×260 magnification.

All pictures and quantifications (done using ImageJ/Fiji v2.0.0-rc-59 software (National Institute of Health, USA)) were performed by a blinded investigator. More precisely, all samples were assigned a random number prior to animal sacrifice, data collection and analysis. At the end of the experiment, the genotype/treatment for each animal was unveiled to allow data comparison and experimental conclusion.

Statistics

Results are reported as mean±SEM. Comparisons between groups were analyzed for significance with the non-parametric Mann-Whitney test or a 2 way ANOVA followed by Sidak's multiple comparison test (for kinetics analyses) using GraphPad Prism v8.0.2 (GraphPad Inc, San Diego, Calif). Differences between groups were considered significant when p≤0.05 (*: p≤0.05; **: p≤0.01; ***: p≤0.001).

### Example 1

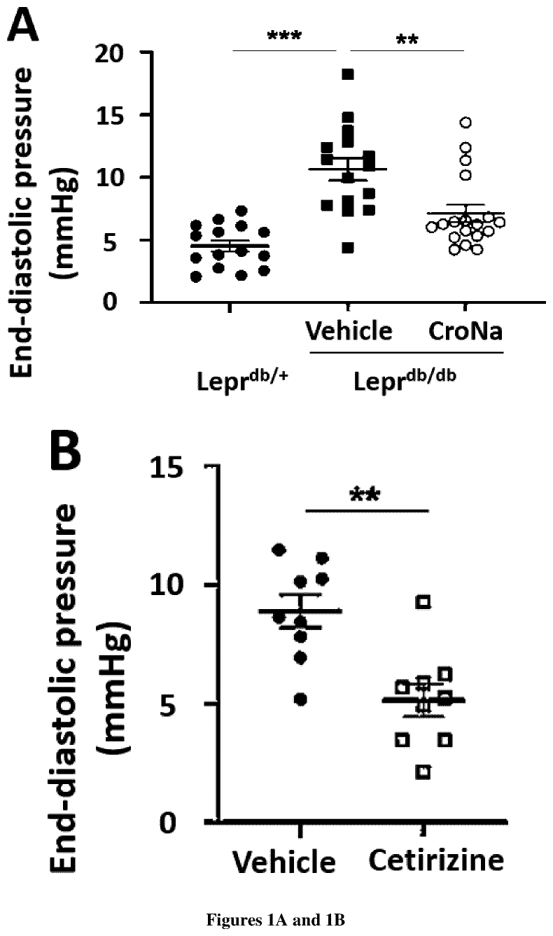

Heart failure with preserved ejection fraction (HFpEF) which results from diastolic dysfunction is a growing epidemiologic problem. However, the pathophysiology of this disease is poorly understood. Our goal is to investigate whether microvessel disease may promote HFpEF. To do so we have used Leptin receptor deficient (Lepr^{db/db}) female mice as a model of HFpEF and performed a transcriptomic analysis via RNA sequencing of the cardiac vascular fraction of both these mice and their control Lepr^{db}/+ littermates. In Lepr^{db/db} female mice, end diastolic pressure (EDP) signing diastolic dysfunction is significantly increased from 3 month of age. It is correlated with a cardiac and cardiomayocyte hypertrophy, vascular leakage, endothelial cell activation and leucocyte infiltration. As expected, the RNA sequencing analysis confirmed endothelial dysfunction. Besides, it also revealed a strong increase in several mast cell markers. We confirmed, via histology, an accumulation of mast cells in the heart of Lepr^{db/db} mice. Importantly, it was associated with increased levels of circulating IgE. Lepr^{db/db} mice were then treated or not with Cromolyn sodium, an inhibitor of mast cell degranulation. After a month treatment, EDP was significantly reduced in Lepr^{db/db} mice demonstrating the critical role of mast cell in the development of diastolic dysfunction in diabetic obese mice (FIG. 1A).

### Example 2

Activated Cardiac Mast Cells, Via Histamine Release, Induce Cardiac Small Vessel Disease in Lepr^{db/db} mice.

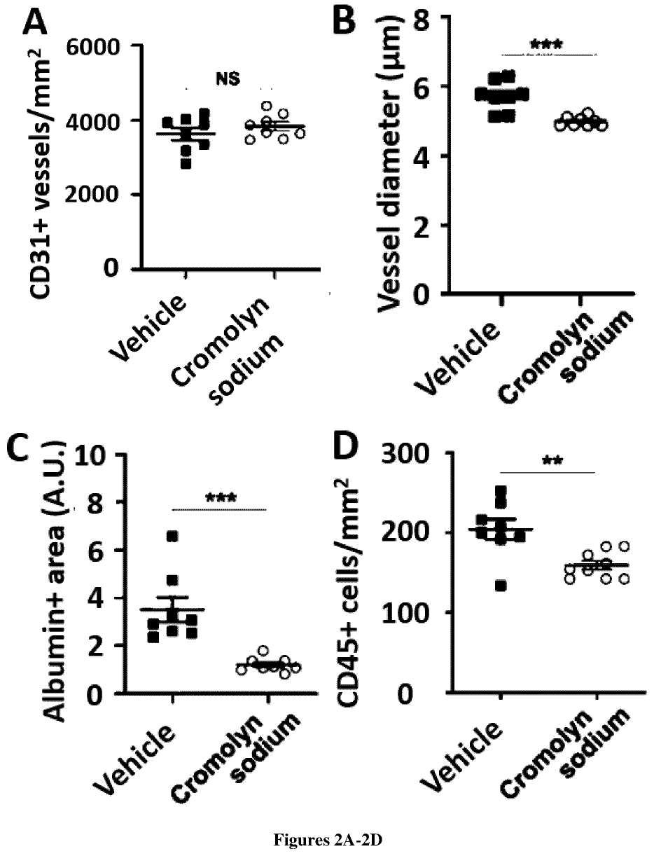

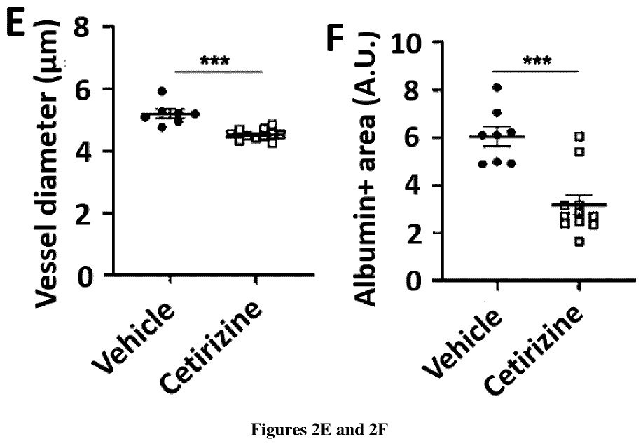

To investigate the role of mast cell degranulation in the pathophysiology of cardiac microvessel disease in Lepr^{db/db} mice, 2 month old Lepr^{db/db} mice (i.e. before they display increased EDP) were treated with 50 mg/Kg/day cromolyn sodium versus vehicle. Mice were sacrificed 28 days later. First, we verified cromolyn sodium therapy was effective and did decrease the percentage of degranulating mast cells in the heart (Data not shown). Then, we investigated the effect of cromolyn sodium therapy on cardiac microvessel phenotype and cardiac inflammation. Cromolyn sodium therapy did not modify cardiac microvessel density (FIG. 2A), however it did reduce capillary diameter (FIG. 2B) and permeability attested by decreased albumin extravasation (FIG. 2C). Moreover, CD45+ leucocyte recruitment was significantly decreased (FIG. 2D).

To summarize, mast cells promote cardiac capillary permeability and vasodilation, which is consistent with the well-known effect of histamine contained in mast cell granules 24. To verify cardiac capillary permeability and vasodilation was indeed due to histamine, 2 month old Lepr^{db/db} mice were treated with 4 mg/kg/days cetirizine versus vehicle. As expected, both the diameter of cardiac capillaries (FIG. 2E) and their permeability (FIG. 2F) were significantly reduced in Lepr^{db/db} mice treated with cetirizine vs vehicle-treated Lepr^{db/db} mice.

In conclusion, mast cells promote cardiac small vessel disease via histamine release in Lepr^{db/db} mice.

Activated Cardiac Mast Cells Promote the Appearance of Diastolic Dysfunction in Lepr^{db/db} Mice.

Finally, to measure to pathophysiological consequences of histamine-induced small vessel disease on cardiac function, we investigated cardiac function in both cromolyn sodium-treated and cetirizine treated-Lepr^{db/db} mice. Ejection fraction, which is normal in Lepr^{db/db} mice was not modulated neither by cromolyn sodium treatment (Data not shown) neither by cetirizine treatment (Data not shown). However, EDP was significantly decreased both cromolyn sodium-treated (FIG. 1A) and cetirizine-treated (FIG. 1B) Lepr^{db/db} mice indicating improved diastolic function. Cromolyn sodium therapy did neither modify the heart weight nor the LV posterior wall thickness nor the mean cardiomyocyte size (Data not shown), indicating that cromolyn sodium therapy does not prevent cardiac hypertrophy.

Altogether these results indicated that mast cells, via secretion of their granule content, promote the development of diastolic dysfunction, however, they do not participate in the development of cardiomyocyte hypertrophy.

Discussion

The present study supports the microvascular hypothesis of HFpEF especially in the setting of obesity and type 2 diabetes. In this paper, we used Lepr^{db/db} female mice as a model of diastolic dysfunction. Lepr^{db/db} female mice have the advantage of recapitulating the main risk factors for HFpEF, i.e. diabetes, obesity female gender and hypertension. Lepr^{db/db} mice were previously shown to display diastolic dysfunction and to recapitulate significant features of human HFpEF. In the present study, we thoroughly characterized the cardiac microvascular phenotype of these mice, notably, via a transcriptomic analysis. Notably we revealed that cardiac microvessel disease is characterized by a decreased capillary density, abnormal vessel permeability and vasoconstriction of arterioles but increased capillary diameter; moreover we showed that ECs display oxidative stress and have a pro-inflammatory and pro-coagulant phenotype. Strikingly, we demonstrated for the first time that, in Lepr^{db/db} mice, cardiac microvessel disease is associated with increased mast cell activation and proved that it participates to the pathophysiology of both cardiac microvessel disease and diastolic dysfunction (Data not shown).

The current paradigm for HFpEF proposes that myocardial remodelling and dysfunction in HFpEF results from the following sequence of events: 1) comorbidities including obesity, diabetes and/or hypertension would induce a systemic low grade pro-inflammatory state; 2) this pro-inflammatory state would induce EC dysfunction characterized by an increased ROS production, a decreased NO synthesis and an increased expression of adhesion molecules such as VCAM-1 and E-selectin; 3) EC dysfunction would lead to a compromised heart perfusion secondary to impaired NO-dependent vasodilatation, oedema and pro-inflammatory/pro-thrombotic phenotype, macrophage infiltration and fibrosis. The present data show that the Lepr^{db/db} female mice model largely recapitulates this paradigm while adding further features. Notably, we demonstrated that mast cell activation, which is either part of the low grade pro-inflammatory state or induced by the low grade inflammatory state of diabetic obese mice, promotes microvascular dysfunction especially vascular permeability and capillary dilation and participates in the development of diastolic dysfunction. However, EC activation may precede mast cell activation (Data not shown). Consistent with a central role of inflammation and microvascular disease in the pathophysiology of HFpEF, Lepr^{db/db} mice do not display significant cardiac fibrosis or major cardiomyocyte abnormalities. Notably, we found that cardiomyocyte hypertrophy does not seem to promote the increased EDP. Indeed, although cromolyn sodium therapy prevents increased EDP, this effect is not associated to cardiomyocyte hypertrophy and dedifferentiation after the onset of diastolic dysfunction, as demonstrated by Myh7 overexpression.

Mast cells are immune cells that reside in the connective tissues including the myocardium. They are characterized by the expression of c-Kit receptors and by their granules containing active mediators including proteases, notably Cma1, Tpsab1 and histamine. Mast cells may be activated by IgEs via their receptor Fcer1a, Complement factors via Toll-like receptors, IgGs or cytokines. They have been associated with several cardiovascular diseases including atherosclerosis, myocardial infarction and aneurysms, pathologies in which mast cells are contributing to the pathogenesis essentially through the release of their granule content. Importantly, circulating Tryptase was recently suggested to be a marker for cardiovascular diseases. Moreover, mast cells have been previously involved in diastolic dysfunction induced by ovariectomy in rats and diabetic cardiomyopathy in streptozotocin-treated mice. The present study thus confirms the significant role of mast cells in cardiovascular diseases. How mast cells are activated in the setting of cardiovascular diseases remains unknown. We found that, in Lepr^{db/db} mice, increased activation of mast cells is associated with increased circulating levels of IgEs. IgE/Fcer1a is the main route of mast cell activation in allergic diseases. Interestingly, IgEs were reported to be elevated in the serum of patients with cardiovascular diseases including coronary arterial disease and myocardial infarction. Consistently, Asthma was shown to be related to an increased incidence of coronary heart disease, particularly in women. More specifically, one study reported that coronary flow reserve, considered as an early marker of endothelial dysfunction is significantly lower in patients with high IgE levels. Altogether, these results further support that Lepr^{db/db} mice are a relevant model of human cardiovascular diseases.

In conclusion, the present study further confirms that inflammation and cardiac microvessel disease are at the heart of HFpEF pathophysiology and identified for the first time mast cells as critical players of cardiac microvessel disease and diastolic dysfunction, making them a promising therapeutic target for HFpEF treatment.

### REFERENCES

Throughout this application, various references describe the state of the art to which this invention pertains. The disclosures of these references are hereby incorporated by reference into the present disclosure.

1. A method of treating heart failure with preserved ejection fraction (HFPEF) in a subject in need thereof comprising administering to the subject a therapeutically effective amount of a mast cell stabilizer.

2. The method of claim 1 wherein the mast cell stabilizer is selected from the group consisting of cromolyn, a dihydropyridine lodoxamide, nedocromil, barnidipine, YC-114, elgodipine, niguldipine, ketotifen, methylxanthines, quercetin, and pharmaceutically salts thereof.

3. The method of claim 1 wherein the mast cell stabilizer is a pharmaceutically acceptable salt of cromolyn.

4. The method of claim 1 wherein the mast cell stabilizer is azatadine, cetirizine, mizolastine, desloratadine, fexofenadine, or levocetirizine.

5. The method of claim 2, wherein the dihydropyridine is nicardipine or nifedipine.

6. The method of claim 3, wherein the pharmaceutically acceptable salt of cromolyn is cromolyn sodium, cromolyn lysinate, ammonium cromonglycate, or magnesium cromoglycate.
